(19) United States
(12) Patent Application Publication
(10) Pub. No.: US 2004/0152665 A1
(43) Pub. Date: Aug. 5, 2004

(54) Amelioration of decreased weight and growth by n-acylated glucosamines

(76) Inventor: Tassos P. Anastassiades, Kingston (CA)

Correspondence Address: MANELLI DENISON & SELTER, 2000 M STREET NW SUITE 700, WASHINGTON, DC, 20036-3307, US

(21) Appl. No.: 10/449,093

(22) Filed: Jun. 2, 2003

(30) Foreign Application Priority Data
Jan. 31, 2003 (CA) 2,417,943

(51) Int. Cl. A61K 31/7008

(52) U.S. Cl. 514/62

(57) ABSTRACT

The invention provides a method of and compositions for enhancing weight gain or growth of a mammal by administering to the mammal an effective amount of a N-acylated-2-glucosamine derivative of the general formula (I).

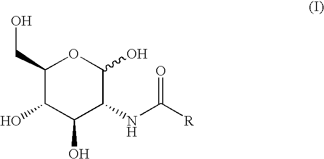

wherein R is an alkyl radical of the general formula CₙH₂ₙ₊₁ and n is selected from 2-12; and pharmaceutically acceptable salts, esters and glucosides thereof. Preferably n is 3.

This application claims priority to Canadian Patent Application No. 2,417,943, filed on Jan. 31, 2003, the complete disclosure of which is incorporated herein by reference.

### FIELD OF THE INVENTION

The invention relates to N-acylated compounds; compositions comprising said compounds; methods of enhancing weight gain and stimulating growth in a mammal by use of said compounds and compositions; and the manufacture of said compositions for said use.

### BACKGROUND TO THE INVENTION

It is well known in the art that medicaments, drugs and substances used in animals or humans in the treatment of diseases, generally show increased toxicity with increased doses administered. The field of toxicology teaches that such medicaments, drugs and substances need to be evaluated for acute or chronic toxicity at increasing doses, administered by a specified route, so that the dose at which adverse effects are observed can be determined. The dose at which half the animals die (LD₅₀), or the acute toxic class method can be employed to evaluate the toxicity of a substance administered to an animal (Diener W et al. Arch Toxicol 1994;68, 599-610).

Glucosamine is an amino-sugar widely distributed in animal tissues (Setnikar I. et al. Arzneimittelforschung 1986 April;36(4):729-35). U.S. Pat. No. 6,479,469 B2—Anastassiades, Tassos P., issued Nov. 12, 2002 and PCT International Application No. PCT/CA01/01217—Anastassiades, Tassos P., published 7 Mar. 2002, teaches the use of compounds shown in general formula I for the treatment of arthritis.

Growth stimulation in animals can be induced by administering peptide growth factors by injection (Imada O et al, Am J Physiol 1987;253, 251-4), For example somatomedin administration affects the nutritional status of dairy cows (Gallo GF, Block E., J Dairy Sci 1990;73, 3266-75). Also enhanced growth in animals can be achieved by prophylactic antibiotic injections (Schunich OC and the m1: Can Vet J 2002; 4:355-62).

### SUMMARY OF THE INVENTION

It is an object of the invention to provide a method of weight gain or enhanced growth of a mammal which is in a normal state of health.

It is a further object to provide a method for enhancing the weight gain or growth of a mammal, whose growth may be retarded, is malnourished or fails to thrive as a result of decreased availability or ingestion of food or feed.

It is a further object to provide a method for enhancing the weight gain or growth of a mammal, whose growth may be retarded, is malnourished or fails to thrive as a result of an illness or condition, or as a result of a combination of illnesses or conditions, or as a result of the treatment of said illnesses or conditions, and which may include decreased availability or ingestion of food or feed.

Accordingly, the invention provides in one broad aspect a method of enhancing the weight gain or growth of a mammal exhibiting decreased weight or growth comprising administering to said mammal an effective amount of a N-acylated-2-glucosamine derivative of the general formula (I):

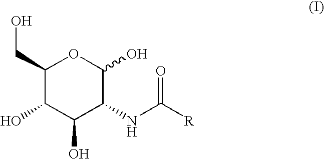

wherein R is an alkyl radical of the general formula CₙH₂ₙ₊₁ wherein n is selected from 2-12, and pharmaceutically acceptable salts, esters and glucosides thereof.

In further defined features, the invention provides a method as hereinabove defined wherein said mammal is in a condition, state or illness selected from the group consisting of

(i) is in a normal state of health;

(ii) has growth which may be retarded, malnourished, or fails to thrive as a result of decreased availability or ingestion of food or feed;

(iii) has growth which may be retarded, malnourished or fails to thrive as a result of an illness of condition, or as a result of a combination of illnesses or conditions, and which may include decreased availability or ingestion of food or feed; and

(iv) has growth which may be retarded, is malnourished or fails to thrive as a result of the treatment or treatments of an illness or treatments of a combination of illnesses or conditions, and which may include decreased availability or ingestion of food or feed.

Preferably, n is selected from 2-5 and more preferably 3.

Thus, more preferred compound is N-butyryl-D-glucosamine, of the formula II:

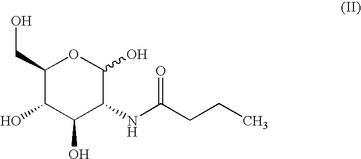

The anomeric and generic structures of formulas (I) and (II), the physical characteristics of the corresponding compounds, the method for their synthesis and tests for purity are described in aforesaid U.S. Pat. No. 6,479,469 B2 and PCT International Application No. PCT/CA01/01217.

The N-acylated derivatives of the general formula I of use in the practise of the invention may be administered to an animal in an effective and adequate non-toxic amount, by typical administrative methods known in the art, for example, by any of the following methods, namely, orally, intravenously, subcutaneously, intramuscularly, trans-dermally or intra-arterially.

The derivatives may be mixed with the food or feed to be ingested by the animal, and/or may be administered as a suitable pharmaceutically acceptable composition in which the active ingredient of use in the practise of the invention is either dissolved or suspended. Solution compositions may be water, salt solutions, other solvents, either alone or in combination with compatible nutrients, antibiotics, pharmaceutical compounds appropriate and suited to the medical condition of the mammal.

Accordingly, in a further aspect, the invention provides a pharmaceutical, nutritional or nutraccutical composition comprising a N-acylated-2-glucosamine derivative of the general formula (I):

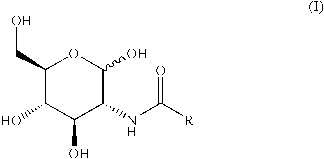

in admixture with a food or feed acceptable for consumption by a mammal, or a pharmaceutically, nutritionally or bacteriologically acceptable carrier or diluent, suitable for oral, rectal, intra-venous, intra-muscular, intra-peritoneal or subcutaneous administration for a treatment or nutritional supplementation of said mammal with a state, condition or illness, as hereinabove defined.

In a yet further aspect, the invention provides a process for the manufacture of a pharmaceutical, nutritional or nutraceutical composition as hereinabove defined, comprising admixing said N-acylated-2-glucosamine derivative with a food or feed or a pharmaceutically, nutritionally or bacteriologically acceptable carrier or diluent suitable for oral, rectal, intravenous, intra-muscular, intra-peritoneal or subcutaneous administration to a mammal.

It will be understood by a person skilled in the art that the active N-acylated glucosamines as hereinbefore defined should be present and administered in respective, effective and sufficient amounts to alleviate or reverse excessive weight loss, failure to grow or thrive, including, but not limited to, symptoms of excessive and undesirable weight loss, as a result or associated with any one, or a combination, of the following conditions, or without a specific cause or condition that can be identified.

Failure to achieve ideal or normal body weight or grow normally or thrive of a mature or immature human or other mammal, as a result of decreased consumption of protein and other necessary dietary components. The failure may be due to decreased availability of the required amount of total food consumption for the animal or the total feed for the animal. Alternately, malnutrition may be as a result of anorexia from any known or unknown cause, including conditions of presumed psychological or psychiatric cause, such as, for example anorexia nervosa, which may occur in presence or absence of the availability of an adequate source of food or feed. Further, the malnutrition, or the failure of the mammal to grow or thrive, may occur in the presence or in the absence of any of the chronic conditions or diseases listed herein below

Failure to achieve ideal or normal body weight or grow normally or thrive of a mature or immature human or other mammal, as a result of a chronic inflammatory condition, such as rheumatoid arthritis or inflammatory bowel disease, treated or untreated with drugs, such as, for example, cortisone.

Failure to achieve ideal or normal body weight or grow normally or thrive of a mature or immature human or other mammal, as a result of a chronic infectious disease such as AIDS, or, a bacterial, fungal or a parasitic disease, or a combination thereof, including, but not limited to the colloquial term “slim disease”, and including the treatments, specifically drug treatments, administered to a mammal suffering from the disease.

Failure to achieve ideal or normal body weight or grow normally or thrive of a mature or immature human or other mammal, as a result of a malignancy, including widespread or advanced cancer, of any type and including treatments, specifically drug treatments, administered to a mammal suffering from the malignancy.

Failure to achieve ideal or normal body weight or grow normally or thrive of a mature or immature human or other mammal, as a result of treatments, specifically drug treatments, for the purposes of suppressing the immune system of the mammal, as for example in organ transplantation.

Failure to achieve ideal or normal body weight or grow normally or thrive of a mature or immature human or other mammal, in association with any combination of chronic and acute diseases, or in the absence of an identifiable association with a disease. Specifically, this includes the ageing process of a mammal occurring in the presence or absence of single or multiple conditions or diseases, identified or suspected, and leading to a wasting state sometimes colloquially referred to as “the dwindles”, or where an other name label, or no name label, is placed on the wasting state.

The prior art teaches that increased weight gain and growth can be also achieved by gene therapy (Dube M G et al., Diabetes 2002 51:1729-36). However, all of these approaches and their mechanisms of action, clearly lead away from the results for the compound of formula II, specifically, and compounds of the general formula I, according to the invention.

It is also known in the prior art that animals that are ingesting a diet relatively deficient in protein can gain weight and grow more rapidly if diets are supplemented so as to increase the protein consumption of the animal (Hastings-Roberts M. M., Zeman FJ. J Nutr 1977 June;107:973-82). However, the results found in the practise of the present invention lead away from the possibility that the compounds of use in the present invention could have resulted in the animals consuming more protein in their diets. This is clearly not the case, since all of the animals used in the present invention test had been made to consume a fixed amount of protein in the diet provided. Also, the possibility that the preferred compound of use in the present invention may have provided an extra source of nitrogen that could account for the observed stimulation of weight gain and growth is rendered non-obvious by the observation that supplementation of the diet with an equimolar, or higher, amount of the parent, glucosamine, led to a relatively decreased rate of growth of the animals. Further, there was a dose response to the weight gain of the animals, so that the maximal amounts of preferred compound, GlcNBu administered did not lead to the maximal growth stimulation.

Although, the amounts of the compounds of general formula (I) that may become available to tissues through the blood stream, after ingestion of these compounds, is not known, pharmacokinetic studies with the aforesaid basic compound, glucosamine, have shown that only very low or not detectable concentrations (Setnikar I et al. Arzneimittelforschung 1986 April;3:729-35) of free glucosamine are found in the circulation after ingestion of this compound.

Humans and other mammals may be malnourished or fail to achieve ideal body weight and children may fail to grow normally, as a result of decreased consumption of protein and other necessary dietary components. Commonly, this is because of decreased availability of the required amount of total food consumption for the human or the total feed for the animal (Charlton KE, Rose D. J Nutr. 2001; 131:2424S-8S). Further, malnutrition may be as a result of anorexia from any known or unknown cause, including presumed psychological or psychiatric causes, such as anorexia nervosa, which may occur in the presence or absence of the availability of an adequate source of food or feed. Also, decreased availability of the required amount of food or feed resulting in malnutrition, or in the failure to grow or thrive, may occur the case in the presence or in the absence of any of the chronic conditions or diseases mentioned hereinbelow. It is believed that there is no prior art with respect to use of the synthetic compounds of general formula I, for situations resulting from decreased availability of the required amount of food or feed resulting in malnutrition, or in the failure to grow or thrive, occurring in the presence or in the absence of any of chronic conditions or diseases.

Growth is often retarded in children with juvenile rheumatoid arthritis (Woo PM, Clin Exp Rheumatol. 1994: Suppl 10:S87-90). Human adults with inflammatory arthritis, such as rheumatoid arthritis, are often suffering from under-nutrition as evidenced primarily by decreased lean body mass and decreased fat mass (Hernandez-Beriain JA et al, Scand J Rheumatol. 1996;25:383-7). The administration of cortisone, a potent anti-inflammatory agent for children with rheumatoid arthritis, can result in a further impairment of and the administration of cortisone to adults with inflammatory arthritis can result in an exacerbation of the catabolic state of these individuals growth (Roubenoff R et al, Am J Clin Nutr 1990;52(6):1113-7). Individuals with other chronic inflammatory diseases, such as inflammatory bowel disease, and so affected children may fail to grow and be malnourished. Prior art includes the use of N-acetyl glucosamine for the treatment of inflammatory conditions (Burger U.S. Pat. No. 5,843,919), but this art does not teach growth or weight gain stimulatory effects of the naturally occurring compound. It is believed that there is no prior art with respect to use of the synthetic compounds of general formula I for retardation of growth or weight gain in reversing the malnutrition in chronic inflammatory conditions.

In chronic infectious diseases, such as AIDS, or, in bacterial, fungal and parasitic diseases adults, children as well as other mammals, suffering from the chronic diseases often are malnourished and lose weight during the advanced stages (Gasparis AP, Tassiopoulos AK, Human Nutrition 2001 Nov-Dec; 17:981-2). Drug treatment itself, including the use of protease inhibitors in AIDS, is believed to contribute further to weight loss and malnutrition. In Africa, where AIDS is endemic, the malnutrition is the most obvious manifestation of the disease, which is colloquially referred to as “slim disease”. It is believed that there is no prior art with respect to use of the synthetic compounds of general formula I for retardation of weight gain or growth or for malnutrition in association with infectious diseases such as AIDS.

Humans and other mammals with a malignancy, including widespread or advanced cancer, of any type, commonly lose weight and become malnourished, and so affected children often fail to grow normally. Commonly, the weight loss or failure to grow is partly or largely attributed to the treatment, specifically the drugs, administered in order to treat the cancer or malignancy, and has been referred to being “iatrogenic” in cause. In other conditions where the treatment is directed toward suppressing the immune system, such as in organ transplantation, drugs similar to those used in cancer therapy, including cortisone, are used and can lead or contribute to iatrogenic weight loss or failure to grow. It is believed that there is no prior art with respect to use of the synthetic compounds of general formula I for the loss of weight or the retardation of growth or for malnutrition in association with advanced or widespread cancers and their drug treatments, or in association of drug treatment for the purpose of suppressing the immune system, either in adults or in children.

Humans and other mammals may suffer from chronic wasting diseases, including multiple diseases of known or unknown causes. The multiple diseases or illnesses may or may not have been diagnosed or identified. These diagnosed or not diagnosed multiple diseases or illnesses occur very commonly in the elderly human, but also in adults and in children, wherein the individuals lose weight and become malnourished. These conditions are often referred to collectively as “failure to thrive”. In the elderly, where declining health, wasting and weight loss is frequently seen, it is common not have one or more clear diagnosis to account for the wasting and weight loss, and an elderly human so affected is sometimes, colloquially referred to as suffering from “the dwindles”. There may be acute diseases or illnesses super-imposed in the chronic wasting diseases resulting in further worsening of the status or the demise of the individual. It is believed that there is no prior art with respect to use of the synthetic compounds of general formula I, for conditions of single or multiple illness of identifiable or unknown cause resulting in wasting, weight loss or for the collective term of “failure to thrive” or for the colloquial term of “the dwindles” (Egbert AM, Nutr Rev 1996;54:S25-30).

Acute toxicology studies were carried out in order to evaluate the toxicity in rats of the aforesaid compounds. Surprisingly, to generally accepted toxicology teaching, I found that no upper limit of feeding the preferred compound, shown in formula II, of the class of compounds of use in the practise of the present invention shown in general formula I, to the animals was reached in terms of observed toxicity. However, surprisingly, I found that there was a specific range of dosages of the preferred compound administered orally, that resulted in the preferred and maximal growth rate and weight gain of the rats. This is in sharp unexpected contrast to the parent compound, namely, glucosamine, which, when fed to the animals in significant amounts led to a decrease in the rate of weight gain and growth, compared to control animals. Further, also feeding the test animals with equimolar amounts of glucose, resulted in no significant change in the rate of weight gain and growth of the animals, as compared to control animals.

Thus, the invention in a further aspect, provides use of a N-acylated-2-glucosamine derivative, as hereinabove defined, or a composition as hereabove defined, suitable for enhancing weight gain or growth in a mammal.

### BRIEF DESCRIPTION OF THE DRAWINGS

In order that the invention may be better understood, preferred embodiments will now be described, by way of example only, with reference to the accompanying drawings, wherein

FIG. 1A is a graph showing the average weight per day for several series of rats fed no or certain supplements;

FIG. 1B is a graph showing the same data as for FIG. 1A, calculated as the average weight gain per day for each condition;

FIG. 2 is a graph showing the averaged weight gained by rats restricted to 10 g of food per day in combination with first dissolved and then solid supplement;

FIG. 3 is a graph showing the average weight gained by rats restricted to 10 g of food per day and gavaged with and without supplements;

FIG. 4 is a graph showing the average weight gained by rats gavaged with various amounts of supplements; and

FIG. 5 represents autoradiagram scans after S³⁵ labelling of epiphyseal growth plates.

### DETAILED DESCRIPTION OF PREFERRED EMBODIMENTS

### Experiment 1

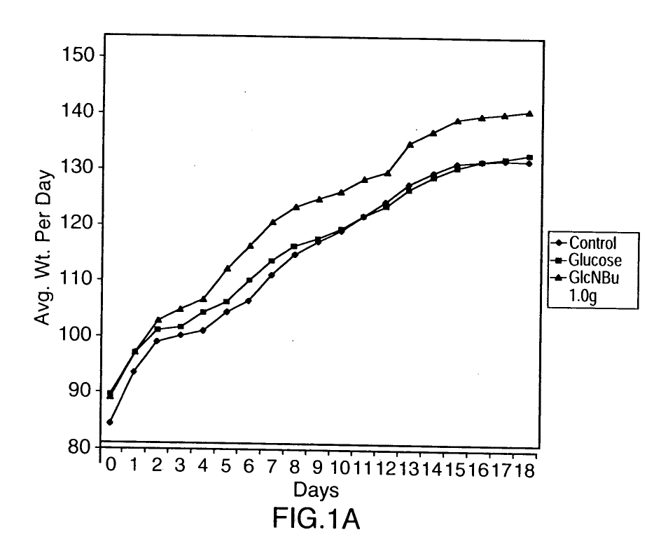

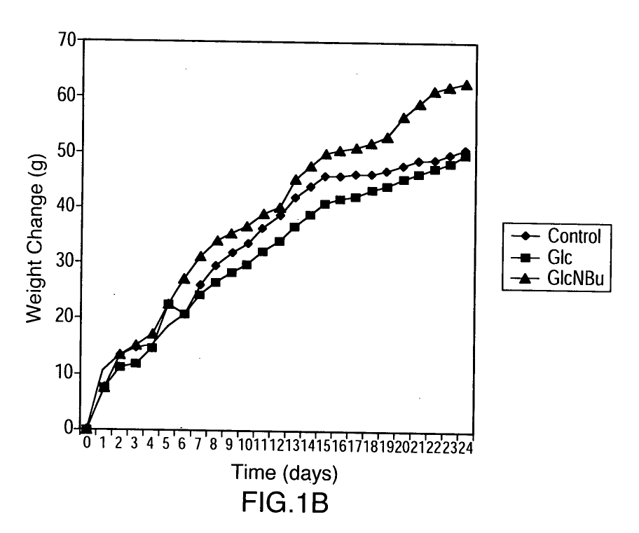

Twelve Sprague-Dawley male rats of body weights between 85 and 90 g each were placed in metabolic cages with access to food, each in an individual separate feeding compartment, so the animals could consume all their food without loss or spillage. Each morning 20 g of crushed commercial chow, which was less than the average determined amount of crushed chow these animals consume. ad lib, was placed in each individual feeding compartment. This procedure ensured that each animal would completely consume all of the crushed chow placed in each feeding compartment over the 24 hours starting with the said placement of the food. Supplementation of the crushed chow with the compounds to be tested was accomplished by mixing the compound with the chow. All test compounds were fully acceptable by the animals and the feeding compartments were emptied by the animals of the crushed chow and any test compound mixed therein, within 24 hr from the beginning of each feeding period. The animals were allowed free access to drinking water throughout the experimental period, which they consumed ad lib. The animals, obtained from a single commercial shipment, were randomly assigned to three experimental groups with four animals in each group. The animals were weighed each morning on day 0 and for each of 18 subsequent consecutive days, as shown in FIG. 1A. The experimental groups of animals are designated in FIG. 1 (A & B) as control; N-butyryl-D-glucosamine, shown as formula (II) and abbreviated as GlcNBu; and D-glucose abbreviated as Glc.

Starting at day 1 and continuing to day 12, inclusive, supplementations of the 20 g of crushed chow per day per animal were as following: Control group—no supplementation; GlcNBu group—supplementation with 1.0 g of GlcNBu per day per animal; Glc—supplementation with 0.75 g of Glc per day per animal, which corresponds to an equimolar amount of this GlcNBu supplementation.

It will be observed from FIG. 1A, that during days 0 to 2, inclusive, there was a rapid and similar average rate of weight gain and growth for all three experimental animal groups. From day 2 to day 4, inclusive, the animals in each of the experimental groups grew at a slower rate, presumably as they adapted to their restricted dietary regimes. However, during this period of time (days 2-4) the GlcNBu experimental group first demonstrated a more rapid average rate of weight gain and growth than either the Glc or the control experimental group. During the time period covering days 4 to 11, the control group had slightly lower average body weights than the Glc supplementation group (the control group having started with slightly lower average weights), but by day 8 the average weights and rates of weight gain and growth of these two groups became essentially identical. By contrast, during the same period of time (days 4-11) the GlcNBu experimental group grew more rapidly than either the Glc or the control group, with the average weights of the GlcNBu being significantly higher than those of the other groups at each of days 4 to 11.

On day 12 and continuing through day 18, the crushed chow (20 g given per day per animal) was further supplemented as follows: Control group—no supplementation; GlcNBu group—supplementation with 1.5 g of GlcNBu per day per animal; Glc —supplementation with 1.148 g of Glc per day per animal, which corresponds to an equimolar amount of this GlcNBu supplementation. It will be observed from FIG. 1 that there was no difference in the average rates of weight gain and growth or weights of the animals in the Glc and the control experimental groups of animals during the period of 12 to 18 days, inclusive. However, for the experimental group of animals receiving the GlcNBu supplementation there was a further increase in the rate and average body weights of the animals, resulting in a still larger differential of the enhancement of weight gain and growth for the GlcNBu group, compared to either the control group or the group receiving the Glc supplementation.

FIG. 1B illustrates the same data for, as in FIG. 1A, calculated as the average weight gain per day for each condition. The significant relative weight gain by the rats fed on the GlcNBu diet, particularly during the last five days of the experimental period, is well illustrated. These results indicate that oral supplementation of the diet by GlcNBu stimulated the weight gain and growth of the rats whose diet was restricted. The increase in weight gain and growth and weight gain could not be accounted by the hexose (glucose) carbon skeleton of GlcNBu, illustrated in Formula (II), since supplementation of the diet by an equimolar amount of glucose as GlcNBu failed to stimulate growth or weight gain in the experimental group so treated.

However, it is possible that the weight gain and growth-stimulatory effect of GlcNBu illustrated in FIGS. 1A and B could be accounted by the glcucosamine (GlcN) moiety, which is part of the structure of GlcNBu shown in Formula (II). In order to ascertain if this might be the case, experiments were performed where GlcN and GlcNBu supplementation were compared, under different sets of conditions, in order to determine the relative effects of these compounds on the weight gain of rats. Also, observations on tolerability and toxicity of the compounds were made.

### Experiment 2

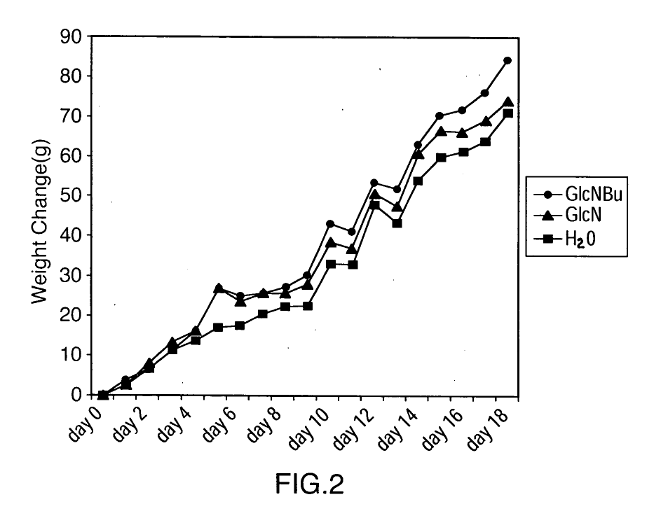

The animals were maintained in metabolic cages for the duration of the study (FIG. 2). Parameters of behavior and physical appearance were observed daily for any indications of toxicological complications. Twelve Sprague Dawley male rats were restricted to 10 g of Purina Rat Chow per day for eight days. Treatment groups were provided with water containing 20 mg/mL GlcN or GlcNBu. Daily volumes of urine produced and water consumed were recorded. The mean water consumption (ml/day) for the three groups was: 22.80, 24.45 and 21.45 for the control, the GlcN-supplemented and the GlcNBu-supplemented groups, respectively. These values were not statistically significant. On day nine of the study, untreated water was provided and the mass of rat chow given daily was increased to 15 g, supplemented with an additional 1.0 g of solid treatment compound. On day 18, the animals were sacrificed by CO₂ asphyxiation and their organs were inspected for damage.

Only the GlcNBu-supplemented animal exhibited consistent statistical differences in weight gained over the water controls, which was maintained after five days of treatment (FIG. 2). Further, the GlcNBu-supplemented group grew somewhat more rapidly than the GlcN group, starting at about day 10 of the experimental time period. Urine volumes and volumes of water consumed were found to be similar for all treatment groups (data not shown). These results suggested that the GlcN moiety could not account for the weight gain and growth-stimulatory effect of GlcNBu. This point was further investigated, with the experimental protocol described below.

### Experiment 3

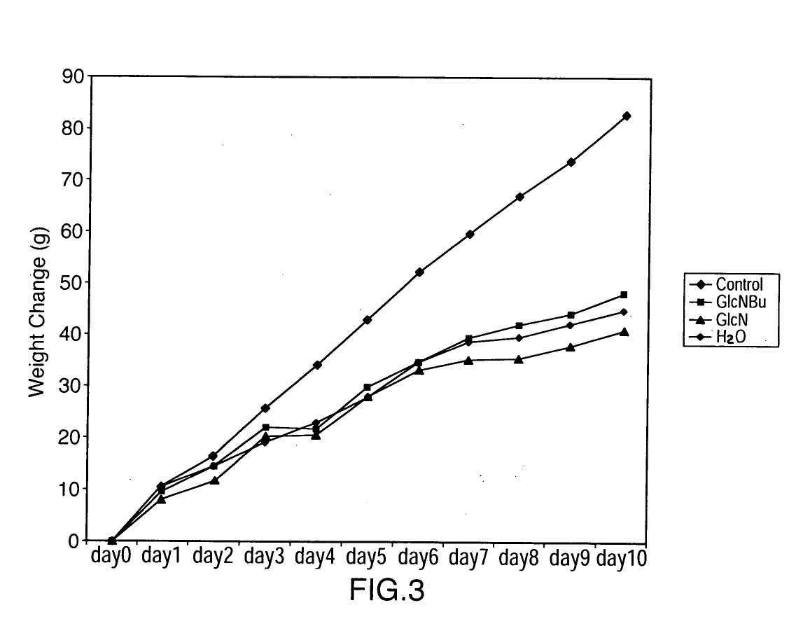

The weights, and behavioral and physical parameters of 20 Sprague Dawley male rats, 5 per treatment group, were monitored over a ten-day period. The control group was given Purina Rat Chow ad libitum. The treatment groups were restricted to 10 g of rat chow per day and were supplemented with one of 300 mg GlcNBu, 300 mg GlcN or water, administered by gavage three times daily (FIG. 3). The animals were then sacrificed by CO₂ asphyxiation and their organs checked for signs of damage.

Average weight gains of rats restricted to 10 g of food and gavaged with either water, GlcN or GlcNBu are depicted in FIG. 3. Supplementation with GlcNBu gives rise to a significantly greater increase in weight over administration of the same amount of GlcN by the eighth day of treatment, under a sequential regimen of free water consumption (with the compounds of interest dissolved in the water), followed by administering said compounds by gavaging the animals.

The gavaging technique was then applied to testing different doses of GlcNBu and comparing the largest dose to an equivalent dose of GlcN, as described below.

### Experiment 4

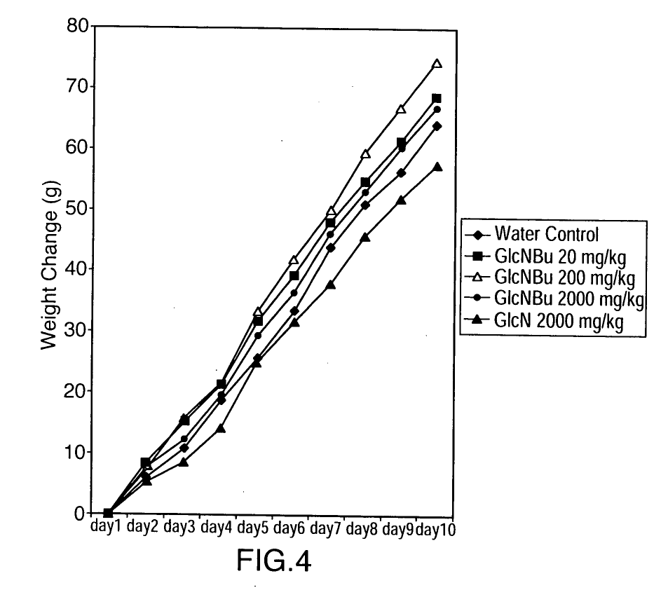

For the data illustrated in FIG. 4, thirty Sprague Dawley male rats were adapted for one week so that each of their weights at the onset of treatment was approximately 185 grams. Each treatment group consisted of six animals, each administered one of the following treatments: water, 20 mg/kg GlcNBu, 200 mg/kg GlcNBu, 2000 mg/kg GlcNBu of 2000 mg/kg GlcN. All animals were fed Purina Rat Chow and water ad libitum. Each morning the animals were weighed and four behavioral and physical parameters were observed for indications of toxicity. For nine days, the compounds were administered using the gavage technique; half doses being administered at 08:00 and at 17:00. Final urine samples were collected, histological examinations were performed to identify any overt signs of damage and then samples from each were harvested. Urinanalysis was performed using reagent strips (Multistix 8 SG from Bayer Inc. Healthcare Division, Etobicoke ON). Animals from all treatment groups displayed levels of activity and curiosity normally seen in healthy animals. No symptoms of neurological damage such as seizures or problems with vision were observed. All animals were well groomed and clean, and their snouts were normal. Histological examinations also failed to reveal any signs of toxicity of GlcN or any of the concentrations of GlcNBu (data not shown). By days 3 and 5, the increase in weights of animals in the 20 mg/kg and 200 mg/kg GlcNBu treatment groups respectively, were statistically significant from those of the water controls. The average weight gain of both the 20 mg/kg and 200 mg/kg GlcNBu treatment groups was also found to be significantly higher than that of the GlcN group. By day 4 the 200 mg/kg GlcNBu group was significantly heavier than the GlcN group. The 20 mg/kg animals began to show significantly more weight gain than the GlcN group during the first six days of treatment, however this margin decreased and became insignificant by day seven. Urinanalysis did not reveal physiologically relevant changes in samples from any of the treatment groups.

These results indicate that there is dose dependency for the stimulatory effect on weight gain and growth by orally administered (gavaging) GlcNBu, which was unexpected. For these experiments, the maximal stimulatory effect was at 200 mg/kg of GlcNBu. Amounts of GlcNBu that were higher (2000 mg/kg) or lower than that (20 mg/kg) resulted in lesser stimulations, which were, nevertheless, higher than the control and the depression of weight gain caused by GlcN administered at 2000 mg/kg.

Weight gain in the animals could occur in the absence of growth. In the following experiment we evaluated the metabolism of the growth plate of animals fed under different conditions, utilizing ³⁵S as the radio-label.

### Experiment 5

At the end of Experiment 1, described above, the rats were injected intra-peritonealy with 20 μCi of ³⁵S[SO₄] in 1 ml of saline on day 18 and sacrificed 20 hours after the injection. The femurs were removed and the distal ends of the bones were processed histologically for autoradiography. Histological sections of the bones were used to make autoradiograms on X-ray film and were also processed for routine histology, including Toluidine blue staining for assessment of the proteoglycans in the growth plate. No histological abnormalities were detected in the histology sections obtained from any of the experimental groups of animals.

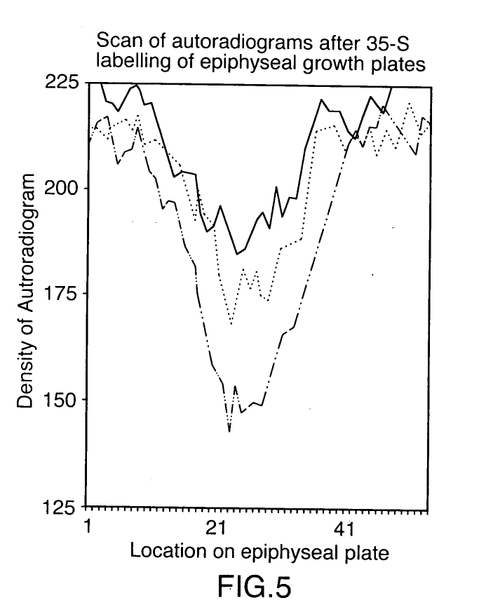

The density of the autoradiograms in the region of the epiphyseal growth plate was evaluated and quantified utilizing a scanning microscope and the appropriate software. The software calculates the density of the autoradiogram by scoring a black region (maximally incorporated radioactivity) as 0 and a white region (no incorporated radioactivity) as 255. The areas under the curves of the scan of the autoradiogram (see FIG. 5) are then computed to give the stated values. The mean values of all of the autoradiograms (12 autoradiograms for each group of animals) were: Control, 125.6; Glc-supplemented, 127.1; GlcNBu—supplemented, 143.2.

FIG. 5 illustrates scans of typical autoradiograms for the control, Glc-supplemented and GlcNBu-supplemented groups. Each scan is illustrated by a curve whose bases (at the top right and left of the figure) represent radioisotope incorporation at the periphery of the epiphyseal growth plate, while the trough of the curve (in the central part of the figure) represents radioisotope incorporation in the central part of the epiphyseal growth plate. It can be seen that for each of the experimental groups less radioactivity has been incorporated in the central part of the epiphyseal growth plates (the trough of the curve), compared to the periphery of the epiphyseal growth plate (the bases of the curve). The curves with the deepest, middle and least troughs represent autoradiograms from the epiphyses of animals fed with GlcNBu (dots-dashes), Glc (dots) and controls (solid line), respectively, as described in Experiment 1.

The overall results indicate that at 20 hours after the ³⁵S[SO₄]injection, the rats whose restricted diet was supplemented with GlcNBu demonstrated less incorporation of the radioisotope, into the sulfated glycosaminoglycans of the proteoglycans of the epiphyseal growth plates, compared to the control or the Glc-supplemented groups. This result is best explained by increased turnover of the proteoglycans of the epiphyseal growth plates of the GlcNBu-supplemented animals, compared to the controls or the Glc-supplemented animals. This increased turnover apparently takes place primarily in the central parts and less so in the periphery of the epiphyseal growth plates. Alternately, the result could be explained by a decreased rate of synthesis of the proteoglycans of the epiphyseal growth plate in the GlcNBu-supplemented animals, compared to the other groups, but this explanation seems less likely.

The results of the autoradiography experiment, taken in conjunction with the measurements of the weights of the animals indicate that supplementing the diet of animals with GlcNBu, resulted in a weight gain of the animals as well as a stimulation of the skeletal growth of said animals. Alternately, the enhancement of weight gain in the GlcNBu-supplemented animals may have been associated with a decrease in skeletal growth, but this seems less likely.

Also, it is noted that there was no difference in weight gain of the animals between the Glc-supplemented group and the control group over the last 4 days of this experiment (FIGS. 1A and 1B), so that the radiolabelling experiment was done during a period of time where there was no difference in the rate weight gain (or loss) between these groups. However, there was also a (relatively small) stimulation of epiphyseal plate metabolism by the Glc-fed group compared to the control group at the end of this time period. These results indicate that stimulation of epiphyseal growth plate metabolism can occur in the absence of weight gain. Obviously, the incremental weight gain of the GlcNBu-fed group was identical (or very similar) when compared to the control or the Glc-fed groups, over the last 4 days of the experiment. On the other hand, the incremental change in the incorporation of the radiolabel into the epiphyseal growth plate for the GlcNBu-fed group was relatively less when compared the Glc-fed group, than when compared to the control group. Thus, feeding GlcNBu to groups of animals resulted in equal weight gains, in spite of the comparator groups demonstrating different extents of stimulation of their growth plate metabolism. It is concluded that the effect of GlcNBu on stimulation of weight gain can not be entirely accounted for by a stimulation of the metabolism of the growth plate of the animals.

The mechanism by which GlcNBu or other N-acyl glucosamine derivatives of general formula (I) may stimulate growth and, or, enhance weight gain is not known and the observations reported above were unexpected.

Although this disclosure has described and illustrated certain preferred embodiments of the invention, it is to be understood that the invention is not restricted to those particular embodiments. Rather, the invention includes all embodiments which are functional or chemical equivalence of the specific embodiments and features that have been described and illustrated.

1. A method of enhancing weight gain or growth of a mammal exhibiting decreased weight or growth, said method comprising of administering to said mammal an effective amount of a N-acylated-2-glucosamine derivative of the general formula (I). 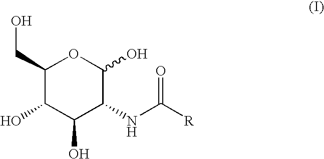
wherein R is an alkyl radical of the general formula CₙH₂ₙ₊₁ wherein n is selected from 2-12; and pharmaceutically acceptable salts, esters and glucosides thereof.

2. A method as defined in claim 1, wherein said mammal is in a condition, state or illness selected from the group consisting of
(i) is in a normal state of health;
(ii) has growth which may be retarded, malnourished, or fails to thrive as a result of decreased availability or ingestion of food or feed;
(iii) has growth which may be retarded, malnourished or fails to thrive as a result of an illness of condition, or as a result of a combination of illnesses or conditions, and which may include decreased availability or ingestion of food or feed; and
(iv) has growth which may be retarded, is malnourished or fails to thrive as a result of the treatment or treatments of an illness or treatments of a combination of illnesses or conditions, and which may include decreased availability or ingestion of food or feed.

3. A method as defined in claim 1 wherein n is 3 and the N-acylated-2-glucosamine has the formula (II):

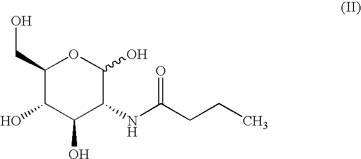

4. A method as defined in claim 2 wherein n is 3 and the N-acylated-2 glucosamine has the formula (II):

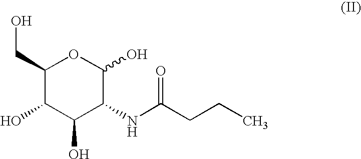

5. A pharmaceutical, nutritional or nutraceutical composition comprising a N-acylated-2-glucosamine derivative of the general formula (I): 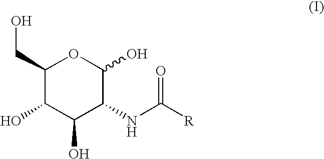
in admixture with a food or feed acceptable for consumption by a mammal, or a pharmaceutically, nutritionally or bacteriologically acceptable carrier or diluent, suitable for oral, rectal, intra-venous, intra-muscular, intra-peritoneal or subcutaneous administration for a treatment or nutritional supplementation of said mammal with a state, condition or illness as defined in claim 1.

6. A pharmaceutical, nutritional or nutraceutical composition as defined in claim 5 comprising a N-acylated-2-glucosamine derivative of the formula (II): [chemical structure]
